(19) United States
(12) Patent Application Publication
(10) Pub. No.: US 2025/0366805 A1
(43) Pub. Date: Dec. 4, 2025

(54) COMPOUND CURVE CABLE CHAIN

(71) Applicant: GLOBUS MEDICAL, INC., Audubon, PA (US)

(72) Inventors: David Cleary, Somerville, MA (US); Norbert Johnson, North Andover, MA (US); Steve Tracy, Litchfield, NH (US); Kevin Zhang, Medford, MA (US)

(21) Appl. No.: 19/306,471

(22) Filed: Aug. 21, 2025

Related U.S. Application Data
(63) Continuation of application No. 18/165,450, filed on Feb. 7, 2023, now Pat. No. 12,396,692.
Application No. 18/165,450 is a continuation-in-part of application No. 17/009,504, filed on Sep. 1, 2020, now Pat. No. 11,571,171.
(60) Provisional application No. 62/904,863, filed on Sep. 24, 2019.

(51) Int. Cl. A61B 6/00; A61B 6/03

(52) U.S. Cl. CPC A61B 6/4441; A61B 6/03; A61B 6/4405

(57) ABSTRACT

Embodiments generally relate to routing a bundle of loose cables with a cable chain during a medical procedure. The cable chain comprises split tubing and a plurality of links extending discretely along a length of the split tubing. Each link comprises a housing including an outer surface and an inner surface. The outer surface comprises a magnet and the inner surface forms a recess in the link. The split tubing is disposed within the recesses of the links.

### CROSS-REFERENCE TO RELATED APPLICATIONS

This patent application is a continuation of a U.S. patent application Ser. No. 18/165,450, filed on Feb. 7, 2023, which is a continuation of a U.S. patent application Ser. No. 17/009,504, filed on Sep. 1, 2020, which claims priority to provisional Patent Application No. 62/904,863 filed on Sep. 24, 2019, all of which are incorporated in their entirety herein.

### BACKGROUND

Healthcare practices have shown a tremendous value of three-dimensional imaging such as computed tomography (CT) imaging. These imaging systems generally contain a fixed bore into which a patient enters from the head or foot. Other areas of care, including the operating room, intensive care departments and emergency departments, rely on two-dimensional imaging (fluoroscopy, ultrasound, 2-D mobile X-ray) as the primary means of diagnosis and therapeutic guidance. While mobile solutions for patient-centric 3-D imaging do exist, they are often limited by their freedom of movement to effectively position the system without moving the patient. Their limited freedom of movement has hindered an acceptance and use of mobile three-dimensional imaging systems.

Therefore, there is a need for mobile three-dimensional imaging systems for use in operating rooms, which can access the patients from any direction or height and produce high quality, three-dimensional images.

### SUMMARY

In an exemplary embodiment, the present disclosure provides a cable chain comprising split tubing and a plurality of links extending discretely along a length of the split tubing. Each link comprises a housing including an outer surface and an inner surface. The outer surface comprises a magnet and the inner surface forms a recess in the link. The split tubing is disposed within the recesses of the links.

In another exemplary embodiment, the present disclosure provides a system comprising a cable chain that may include split tubing and a plurality of links extending discretely along a length of the split tubing. Each link may include a housing comprising an outer surface and an inner surface. The outer surface may comprise a magnet and the inner surface may form a recess in each of the links. The split tubing is disposed within the recesses of the links. A bundle of cables may be disposed within the split tubing. The system may also include a gantry. The cable chain may be movably disposed within the gantry.

In another exemplary embodiment, the present disclosure provides a system comprising first and second cable chains. Each cable chain may comprise split tubing and a plurality of links extending discretely along a length of the split tubing. Each link may comprise a housing including an outer surface and an inner surface. The outer surface may comprise a magnet and the inner surface may form a recess in the link. The split tubing is disposed within the recesses of the links. The system may also include a gantry comprising first and second sidewalls. The first cable chain is movably disposed along the first sidewall, and the second cable chain is movably disposed along the second sidewall.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

These drawings illustrate certain aspects of some of the embodiments of the present disclosure and should not be used to limit or define the disclosure.

FIG. 1 is a perspective rear view of an imaging system, in accordance with some embodiments of the present disclosure;

FIG. 2 is a schematic diagram of an imaging controller system, in accordance with some embodiments of the present disclosure.

FIG. 3 is a perspective front view of the imaging system, in accordance with some embodiments of the present disclosure;

FIG. 4 is a perspective view of the imaging system in which the gantry has been rotated about the X-axis by 90°, in accordance with some embodiments of the present disclosure;

FIGS. 5-7 illustrate perspective views of the gantry with a cabling arrangement, in accordance with some embodiments of the present disclosure;

FIG. 8 illustrates a motor assembly for telescopically controlling the C-arms of the gantry, in accordance with some embodiments of the present disclosure;

FIGS. 9A-9G illustrate perspective views of a 360° rotation of the gantry in 60° increments, in accordance with some embodiments of the present disclosure;

FIG. 10 illustrates a perspective view of a cable chain, in accordance with embodiments of the present disclosure;

FIG. 11 illustrates a perspective view of the tubing and a link of the cable chain in accordance with embodiments of the present disclosure;

FIGS. 12A and 12B illustrate perspective views of the tubing assembled around the cable bundle, in accordance with some embodiments of the present disclosure;

FIG. 13 illustrates a cross-sectional front view depicting the cable chain, in accordance with some embodiments of the present disclosure;

FIG. 14 illustrates a cross-sectional front view depicting a nested geometry of multiple cable chains movably disposed along a gantry; and

FIG. 15 illustrates a perspective view of the cable chain movably disposed against the gantry, in accordance with embodiments of the present disclosure.

### DETAILED DESCRIPTION

For the purposes of promoting an understanding of the principles of the present disclosure, reference will now be made to the implementations illustrated in the drawings and specific language will be used to describe them. It will nevertheless be understood that no limitation of the scope of the disclosure may be intended. Any alterations and further modifications to the described devices, instruments, methods, and any further application of the principles of the present disclosure are fully contemplated as would normally occur to one skilled in the art to which the disclosure relates. In particular, it may be fully contemplated that the features, components, and/or steps described with reference to one or more implementations may be combined with the features, components, and/or steps described with reference to other implementations of the present disclosure. For simplicity, in some instances the same reference numbers are used throughout the drawings to refer to the same or like parts.

Embodiments generally relate to a cable chain used to manage dynamic cable bundles routed through geographic information system (GIS) telescoping C-gantries (“gantries”). The cables may be routed internally through the gantries to facilitate 360° scanning of a patient. Routing cables externally may risk entangling the patient or a patient table during a 360° scan. Therefore, an internal cable management system is desired. More particularly, the cable chain may trace a compound curve rather than a planar bend (i.e., a path of the cable chain is curved when viewed from two planes). Further, the cable chain may operate in any orientation relative to gravity and may utilize a magnetic preload for attachment to the gantry. Additionally, the cable chain may have a low cross-sectional profile and may be able to turn along a tight bend radius to allow operation of the cable chain within a limited volume inside the gantry. The cable chain may ensure that the cable bundle follows a prescribed path and may also protect the cables through a full range of motion along the gantry. Absent a cable management system, the cable bundle may be prone to damage, sag under its own weight, intermittently causing interference issues, and/or jamming. These issues may be exacerbated depending on a tilt orientation of the gantry relative to gravity.

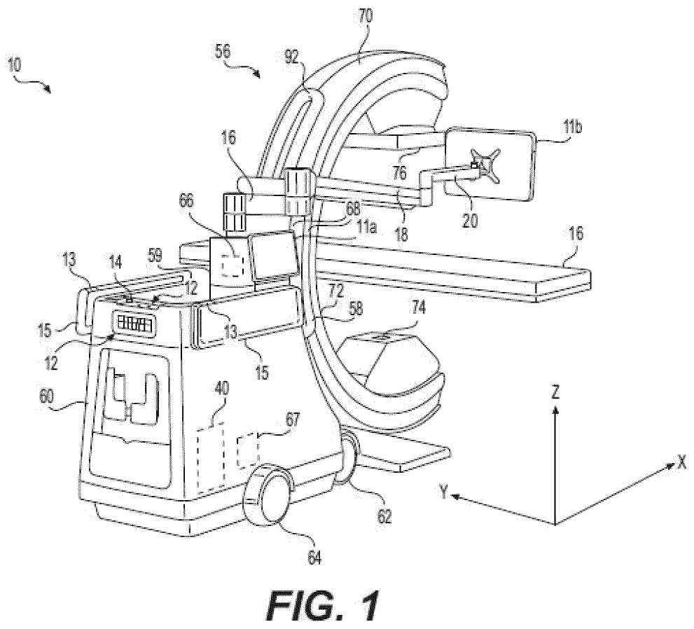

FIG. 1 is a schematic diagram showing an imaging system 10, such as a computerized tomographic (CT) x-ray scanner, in accordance with embodiments of the present disclosure. The imaging system 10 may include a movable station 60 and a gantry 56. The movable station may include a vertical shaft 59 and a gantry mount 58 which may be rotatably attached to the vertical shaft. The movable station 60 may include two front omni-directional wheels 62 and two rear omnidirectional wheels 64, which together may provide movement of the movable station 60 in any direction in an X-Y plane. The omni-directional wheels 62,64 may be obtained, for example, from Active Robots Limited of Somerset, U.K. A pair of handles 13 mounted to the housing of the movable station 60 may allow a user to manually maneuver the station 60. A motor 66 attached to the vertical shaft 59 may rotate the gantry mount 58 360° about the X-axis, and a motor 67 may move the gantry mount 58 vertically along the z-axis under the control of the control module 51. The gantry 56 may include a first C-arm 70 slidably coupled to the gantry mount 58 and a second C-arm 72 which may be slidably coupled to the first C-arm. In the embodiment shown, the first and second C-arms 70,72 are outer and inner C-arms, respectively. In the embodiment shown, the outer and inner C-arms 70,72 are circular in shape and rotate circumferentially about a central axis so as to allow imaging of a patient who is lying in bed 16 without the need to transfer the patient.

An imaging signal transmitter 74 such as an X-ray beam transmitter may be mounted to one side of the second C-arm 72 while an imaging sensor 74 such as an X-ray detector array is mounted to the other side of the second C-arm and faces the transmitter. In operation, the X-ray transmitter 74 transmits an X-ray beam which is received by the X-ray detector 76 after passing through a relevant portion of a patient (not shown). In one embodiment, the system 10 may be a multi-modality x-ray imaging system designed with surgery in mind. The three imaging modalities include fluoroscopy, 2D Radiography, and Cone-beam CT. Fluoroscopy is a medical imaging technique that shows a continuous X-ray image on a monitor, much like an X-ray movie. 2D Radiography is an imaging technique that uses X-rays to view the internal structure of a non-uniformly composed and opaque object such as the human body. CBCT (cone beam 3D imaging or cone beam computer tomography) also referred to as C-arm CT, is a medical imaging technique consisting of X-ray computed tomography where the X-rays are divergent, forming a cone. The movable station 60 may include an imaging controller system 40 which serves a dual function of (1) controlling the movement of the omni-directional wheels 62,64, gantry mount 58 and the gantry 56 to position the imaging signal transmitter 74 in relation to the patient, and (2) controlling imaging functions for imaging the patient once the gantry 56 has been properly positioned.

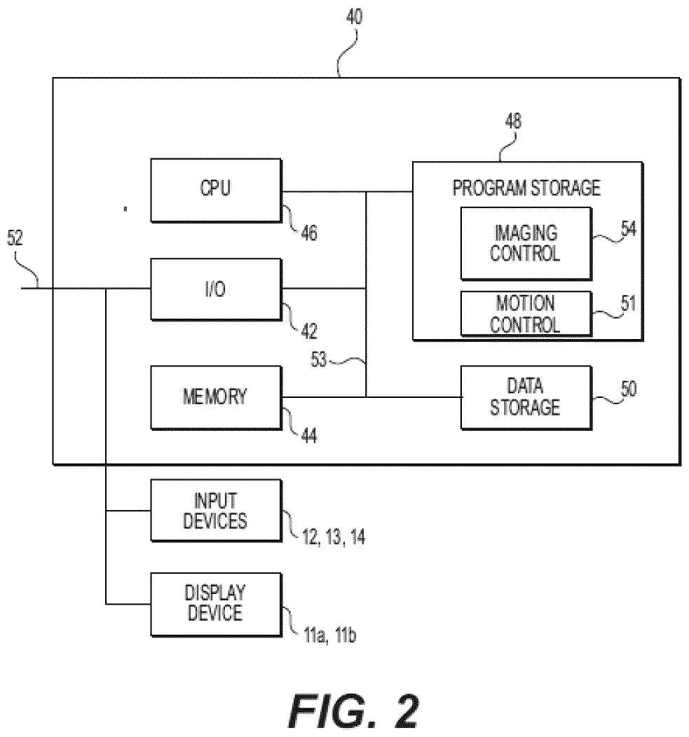

FIG. 2 illustrates the imaging controller system 40 connected to a communication link 52, in accordance with embodiments of the present disclosure. The imaging controller system 40 may be connected to the communication link 52 through an I/O interface 42 such as a USB (universal serial bus) interface, which receives information from and sends information over the communication link 52. The imaging controller system 40 includes memory storage 44 such as RAM (random access memory), processor (CPU) 46, program storage 48 such as ROM or EEPROM, and data storage 50 such as a hard disk, all commonly connected to each other through a bus 53. The program storage 48 stores, among others, imaging control module 54 and motion control module 51, each containing software to be executed by the processor 46. The motion control module 51 executed by the processor 46 controls the wheels 62,64 of the movable station 60 and various motors in the gantry mount 58 and gantry 56 to position the station 60 near the patient and position the gantry in an appropriate position for imaging a relevant part of the patient. The imaging control module 54 executed by the processor 46 controls the imaging signal transmitter 74 and detector array 76 to image the patient body. In one embodiment, the imaging control module images different planar layers of the body and stores them in the memory 44. In addition, the imaging control module 54 can process the stack of images stored in the memory 44 and generate a three-dimensional image. Alternatively, the stored images can be transmitted to a host system (not shown) for image processing.

The motion control module 51 and imaging control module 54 may include a user interface module that interacts with the user through the display devices 11a and 11b and input devices such as keyboard and buttons 12 and joystick 14. Strain gauges 13 mounted to the handles 15 may be coupled to the I/O device 42 and conveniently provide movement of the movable station 12 in any direction (X, Y, Wag) while the user is holding the handles 15 by hand as will be discussed in more detail below. The user interface module assists the user in positioning the gantry 56. Any of the software program modules in the program storage 48 and data from the data storage 50 can be transferred to the memory 44 as needed and is executed by the CPU 46. The display device 11a is attached to the housing of the movable station 60 near the gantry mount 58 and display device 11b is coupled to the movable station through three rotatable display arms 16, 18 and 20. First display arm 16 is rotatably attached to the movable station 60, second display arm 18 is rotatably attached to the first arm 16 and third display arm 20 is rotatably attached to the second display arm. The display devices 11a,11b can have touch screens to also serve as input devices through the use of user interface modules in the modules 51 and 54 to provide maximum flexibility for the user.

Navigation markers 68 placed on the gantry mount 58 may be connected to the imaging controller system 40 through the link 52. Under the control of the motion control module 51, the markers 68 allow automatic or semi-automatic positioning of the gantry 56 in relation to the patient bed or OR (operating room) table via a navigation system (not shown). The markers 68 may be optical, electromagnetic or the like. Information can be provided by the navigation system to command the gantry 56 or system 10 to precise locations. One example may be that a surgeon holding a navigated probe at a desired orientation that tells the imaging system 10 to acquire a Fluoro or Radiographic image along that specified trajectory. Advantageously, this may remove the need for scout shots thus reducing x-ray exposure to the patient and OR staff. The navigation markers 68 on the gantry 56 may also allow for automatic registration of 2D or 3D images acquired by the system 10. The markers 68 may also allow for precise repositioning of the system 10 in the event the patient has moved.

In the embodiment shown, the system 10 may provide a large range of motion in all 6-degrees of freedom (“DOF”). Under the control of the motion control module 51, there are two main modes of motion: positioning of the movable station 60 and positioning of the gantry 56. The movable station 60 positioning is accomplished via the four omni-directional wheels 62,64. These wheels 62,64 allow the movable station 60 to be positioned in all three DOF about the horizontal plane (X, Y, Wag). “Wag” is a system 10 rotation about the vertical axis (Zaxis), “X” is a system forward and backward positioning along the X-axis, and “Y” is system 10 lateral motion along the Y-axis. Under the control of the control module 51, the system 10 can be positioned in any combination of X, Y, and Wag (Wag about any arbitrary Z-axis due to use of omnidirectional wheels 62,64) with unlimited range of motion. In particular, the omni-directional wheels 62,64 allow for positioning in tight spaces, narrow corridors, or for precisely traversing up and down the length of an OR table or patient bed.

The gantry 56 positioning may be accomplished about (Z, Tilt, Rotor). “Z” is gantry 56 vertical positioning, “Tilt” is rotation about the horizontal axis parallel to the X-axis as described above, and “Rotor” is rotation about the horizontal axis parallel to the Y-axis as described above. Together with the movable station 60 positioning and gantry 56 positioning, the system 10 provides a range of motion in all 6 DOF (X, Y, Wag, Z, Tilt and Rotor) to place the movable station 60 and the imaging transmitter 74 and sensor 76 precisely where they are needed. Advantageously, 3-D imaging can be performed regardless of whether the patient is standing up, sitting up or lying in bed and without having to move the patient. Precise positions of the system 10 can be stored in the storage memory 50 and recalled at any time by the motion control module 51. This is not limited to gantry 56 positioning but also includes system 10 positioning due to the omni-directional wheels 62,64.

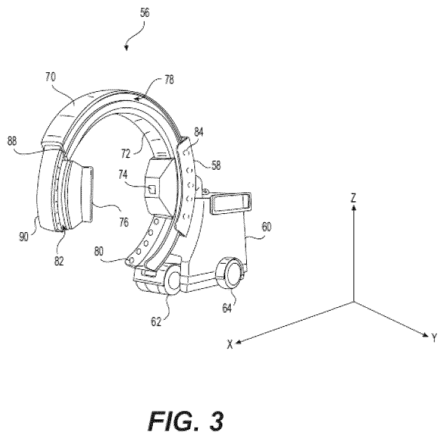

FIG. 3 is a perspective front view of the imaging system of FIG. 1, in accordance with embodiments of the present disclosure. Each of the gantry mount 58, outer C-arm 70 and inner C-arm 72 respectively has a pair of side frames 86, 88,90 that face each other. A plurality of uniformly spaced rollers 84 are mounted on the inner sides of the side frames 86 of the gantry mount 58. The outer C-arm 70 has a pair of guide rails 78 on the outer sides of the side frames 88. The rollers 84 are coupled to the guide rails 78. As shown, the rollers 84 and the guide rails 78 are designed to allow the outer C-arm 78 to telescopically slide along the gantry mount 58 so as to allow at least 180 degree rotation of the C-arm about its central axis relative to the gantry mount. A plurality of uniformly spaced rollers 80 are mounted on the inner sides of the side frames 88 of the outer C-arm 70. The inner C-arm 72 has a pair of guide rails 82 on the outer sides of the side frames 90. The rollers 80 are coupled to the guide rails 82. As shown, the rollers 80 and the guide rails 82 are designed to allow the inner C-arm 72 to telescopically slide along the outer C-arm 70 so as to allow at least 180 degree rotation of the C-arm about its central axis relative to the outer C-arm. Thus, the present invention as disclosed herein advantageously allows the gantry 56 to rotate about its central axis a full 360 degrees to provide the maximum flexibility in positioning the imaging system 10 with minimum disturbance of the patient. In another aspect of the present invention, a unique cabling arrangement is provided to make the imaging system 10 more compact and visually more appealing.

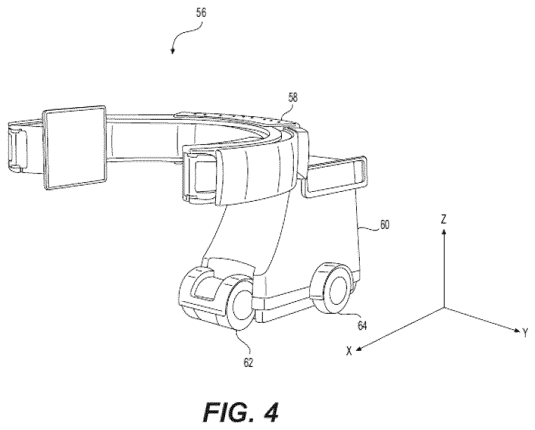

FIG. 4 is a perspective view of the imaging system 10, in accordance with embodiments of the present disclosure. As illustrated, the gantry 56 has been rotated about the X-axis by 90°. Also illustrated are the movable station 60 and the gantry mount 58 for the gantry 56. The movable station 60 may include two front omni-directional wheels 62 and two rear omnidirectional wheels 64, which together may provide movement of the movable station 60 in any direction in an X-Y plane.

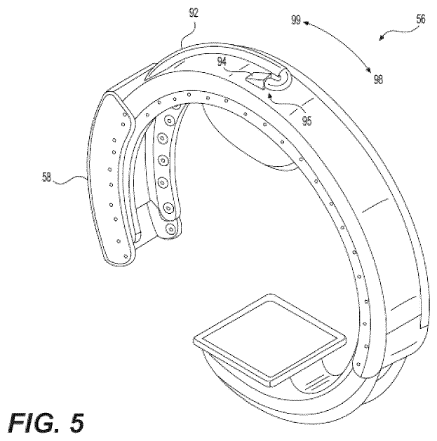

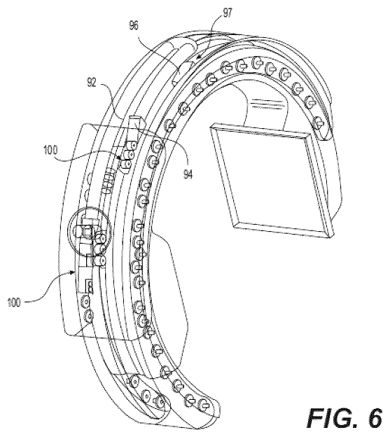

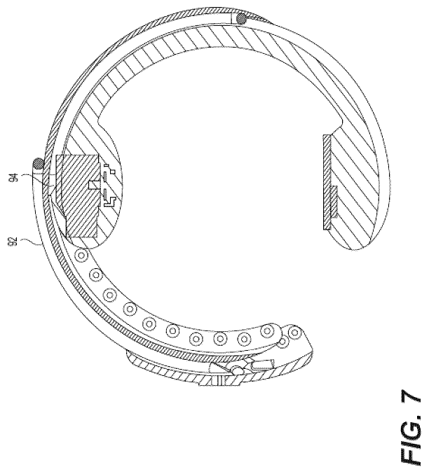

FIGS. 5-7 illustrate a cable carrier/harness 92, in accordance with embodiments of the present disclosure. The cable carrier/harness 92 may contain electrical cables to carry signals between the imaging controller system 40 and various motors, X-ray transmitter 74, imaging sensor 76 and various electronic circuits in the gantry 56. A first cable router 94 is mounted to the outer surface of the outer C-arm 70 and a second cable router 96 is mounted to the outer surface of the inner C-arm 72. Each cable router 94,96 has a through-hole 95,97 through which the cable carrier 92 passes. The cable carrier 92 extends from the gantry mount 56 over the outer surface of the first C-arm 70, through the through-hole 95 of the first cable router 94 and over an outer surface of the second C-arm 72. The cable carrier 92 overlying the first C-arm 70 extends in a first circumferential direction (clock-wise as shown) 98 and enters the first cable router 94 in a second circumferential direction (counter clock-wise as shown) 99 opposite to the first circumferential direction to create a 180 degree service loop over the outer surface of the first C-arm. From there, the cable carrier 92 extends in the first circumferential direction 98 and enters the second cable router in the second circumferential direction 99 to create another service loop over the outer surface of the second C-arm 72. The particular locations of the first and second cable routers 94,96 combined with the service loops allow slack in the cable carrier 92 to provide the gantry 56 with full 360 degrees rotation without tangling or causing stress in the cable carrier. In the embodiment shown, the routers are mounted near the midpoint of the C-arms.

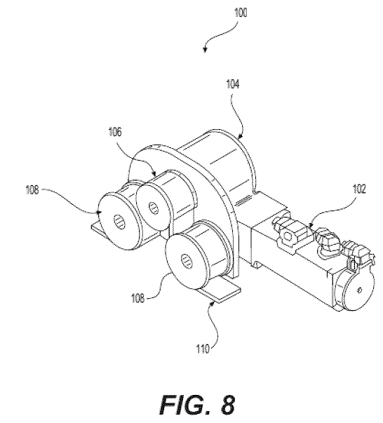

FIG. 8 illustrates one embodiment of a motor assembly 100 that could be used to telescopically rotate the outer C-arm 70 relative to the gantry mount 58 and inner C-arm 72 relative to the outer C-arm. Each motor assembly 100 includes a servo motor 102 with encoder feedback, gear box 104 to change the turning ratio, drive pulley 106, idler pulleys 108 and belt 110 threaded between the drive pulley and the idler pulleys. One motor assembly 100 is mounted to the gantry mount to move the outer C-arm 70 relative to the gantry mount and another motor assembly is mounted to the outer C-arm 70 near the center of the arm to move the inner C-arm 70 relative to the outer C-arm.

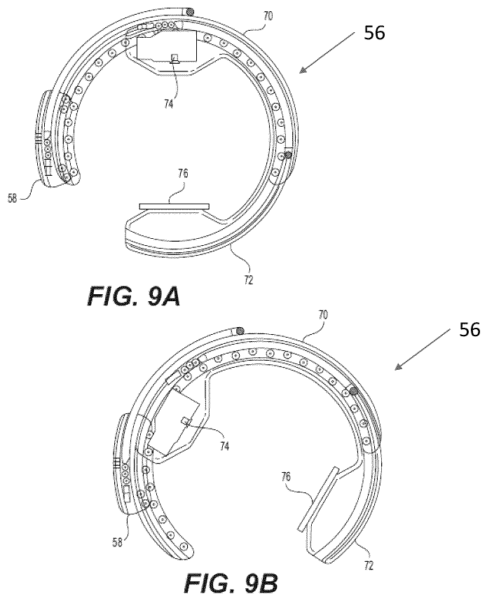

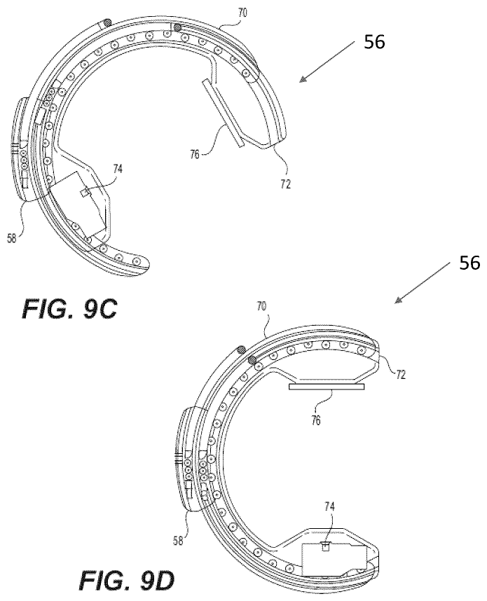

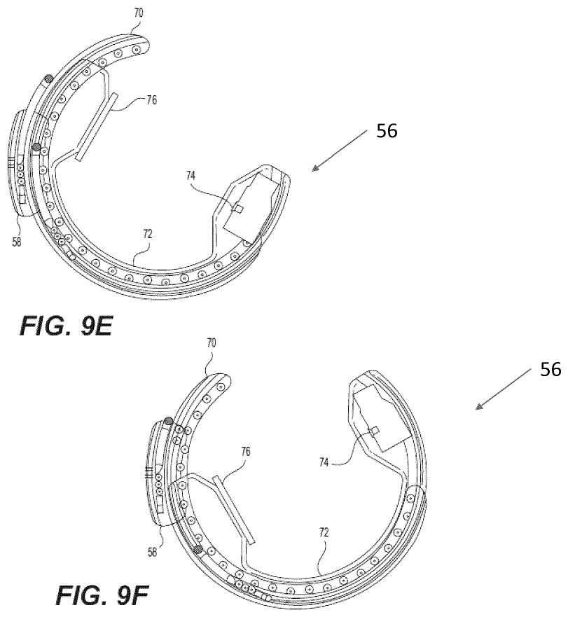

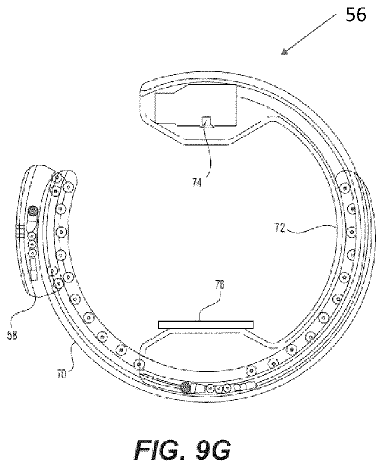

FIGS. 9A-9G illustrate 360° rotation of the gantry 56 in the counterclockwise direction in 60° increments with FIG. 9A representing a 0° position of the imaging sensor 76 and transmitter 74. FIG. 9B represents a 60° turn/position of the gantry 56. FIGS. 9C-9F illustrate further movement of the gantry 56. For each 60° turn of the gantry 56, the motor assemblies 100, under the control of the motion control module 51, turn the inner C-arm 72 by 30° counter-clock wise and also turn the outer C-arm 70 by 30° counter-clock wise for a combined 60° turn. FIG. 9G represents a full 360° turn of the gantry 56. As can be seen, the outer C-arm 70 and inner C-arm 72 have each moved 180° from the original 0° position of FIG. 9A.

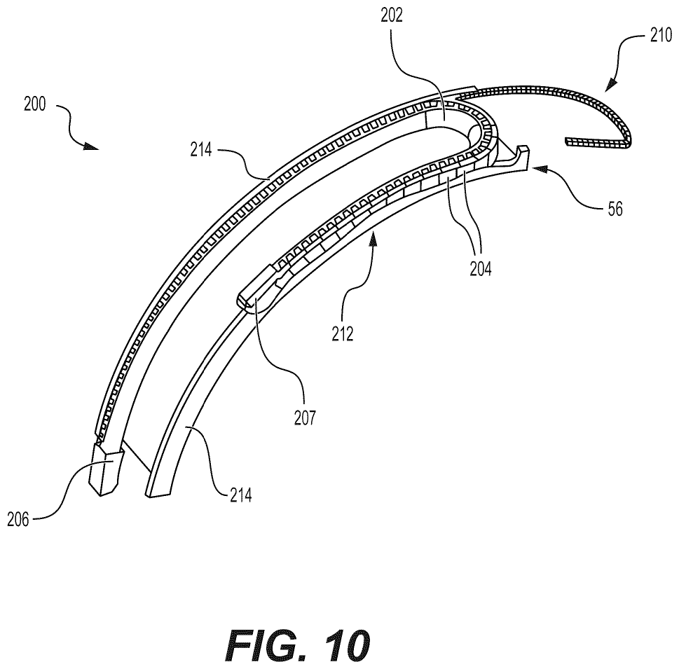

FIG. 10 illustrates a perspective view of a GIS cable chain 200 (“chain 200”) in accordance with some embodiments of the present disclosure. The chain 200 may include corrugated tubing 202 (“tubing 202”) and carrier links 204 (“links 204”) that may be coaxially aligned and positioned discretely along a length of the tubing 202. The tubing 202 may be made of a flexible material such as plastic or rubber. The tubing 202 may contain or encompass an unbound or loose cable bundle (not shown). The links 204 may fit over or encompass at least a portion of the tubing 202. The links 204 may extend longitudinally along the tubing 202 and directly abut one another in an end-to-end configuration, however, the links 204 are not rigidly hinged together, to allow the chain 200 to leverage flexibility of the tubing 202 and achieve a desirable or minimum bend radii. The chain 200 may include a first end clamp 206 that may be fixed to a stationary gantry mount (e.g., the gantry mount 58 shown on FIG. 1). The chain 200 may also include a second end clamp 207 opposite to the first end clamp 206. The second end clamp 207 may be attached to a moving gantry rolling interface or sidewall(s) 214 of the gantry 56 (also shown on FIG. 1 for example). The chain 200 may slide or otherwise move along the sidewalls 214 of the gantry 56. Compound curves 210 and 212 represent different configurations or prescribed travel paths of the chain 200, for example.

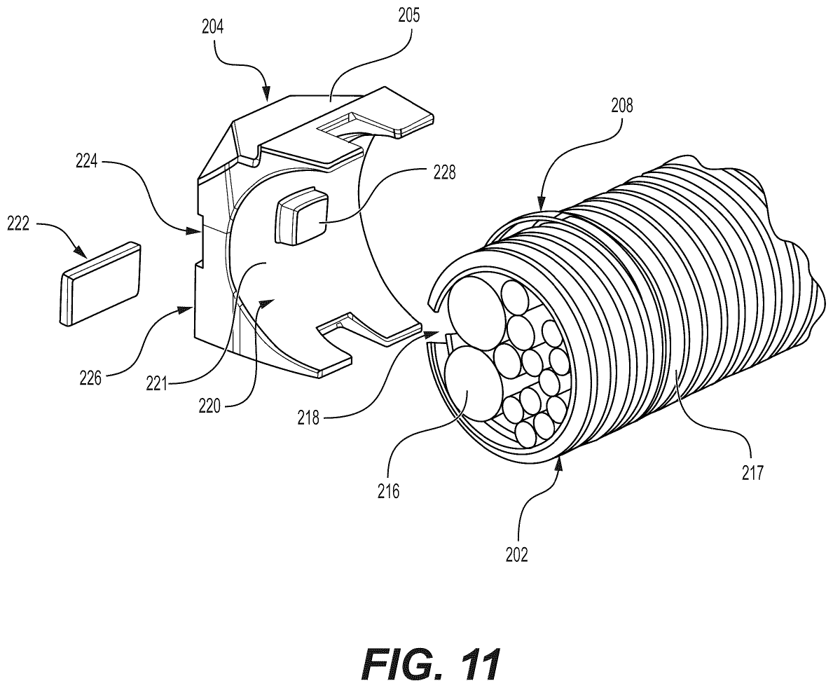

FIG. 11 illustrates a perspective view of the tubing 202 and a link 204 in accordance with some embodiments of the present disclosure. As illustrated, an outer surface of the tubing 202 may include ribs or corrugations 217 that extend along a circumference of the tubing 202, in some examples. The tubing 202 may encompass a cable bundle 216. In some embodiments, the tubing 202 may be split. For example, a slit or gap 218 may extend longitudinally along a wall of the tubing 202 (e.g., split tubing) to allow disposal of the cable bundle 216 within the tubing 202, for example. That is, the gap 218 is in fluid communication with an interior of the tubing 202. In some examples, a wire tie 208 may secure the tubing 202 against the bundle 216, by tightening the wire tie 208, for example. The wire tie 208 may be disposed between two corrugations 217, for example. Each link 204 may each include a housing 205. The housing 205 may include an inner surface or recess 220 that partially encompasses the tubing 202. The recess 220 may be bound by a contoured or curved inner surface 221 to contact and receive the tubing 202. The contours or curves of the tubing 202 and the recess 220 of the link 204 may complement each other to ensure a snug fit of the tubing 202 against the recess 220 of the link 204.

In some embodiments, a permanent magnet 222 may be embedded into an exterior slot 224 of the housing 205 of each link 204 such that the magnet 222 is flush with an outer or exterior surface 226 of the link 204 to protect the magnet 222 from damage. Each link 204 may be magnetically attracted to ferrous material in the sidewall 214 (e.g., shown on FIG. 10) which may ensure that the chain 200 (shown on FIG. 10, for example) is preloaded into the sidewall 214. The magnet 222 also assists in constraining a chain orientation of the chain 200 (e.g., shown on FIG. 10) when each link 204 rolls from one sidewall 214 (e.g., shown on FIG. 10) to another sidewall 214 of the gantry 56 (e.g., shown on FIG. 1). In some embodiments, each magnet 222 may be sized to support a weight of the chain 200 against gravity when the chain 200 or a portion thereof, is in a horizontal orientation.

Each link 204 may be constrained rotationally around the tubing 202 with a tab 228 that protrudes from the recess 220 of each link 204. For example, the tab 228 may extend into the gap 218 (e.g., split portion) of the tubing 202 to prevent rotation of the tubing 202 relative to the tab 228. The tab 228 and the sidewall 214 (e.g., shown on FIG. 10) may ensure that the tubing 202 resists undesired torsional motion or twisting. The tab 228 may also serve as an attachment point for the tubing 202, in some examples.

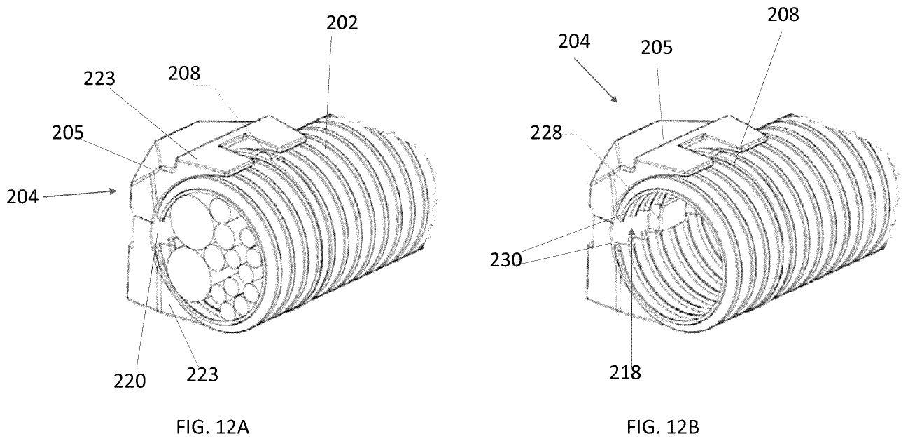

FIG. 12A illustrates a perspective view of the tubing 202 assembled around the cable bundle 216 and the link 204 assembled around the tubing 202, in accordance with some embodiments of the present disclosure. The wire tic 208 may extend around or along a circumference of the tubing 202. The tubing 202 may be disposed within the recess 220 of the link 204. The link 204 may include extended portions 223 that may partially form the recess 220 and partially wrap around the tubing 202 to assist in securing the tubing 202.

FIG. 12B illustrates a perspective view of the tab 228 protruding from the recess 220 of the link 204, in accordance with some embodiments of the present disclosure. The tab 228 is secured within the gap 218. The gap 218 may be positioned between edges 230 of the tubing 202. The edges 230 may extend along the length of the tubing 202 and squeeze the tab 228 to secure the tubing 202 to the link 204, in some examples. The wire tie 208 may be tightened to compress or squeeze the edges 230 against the tab 228 to secure the tubing to the link 204.

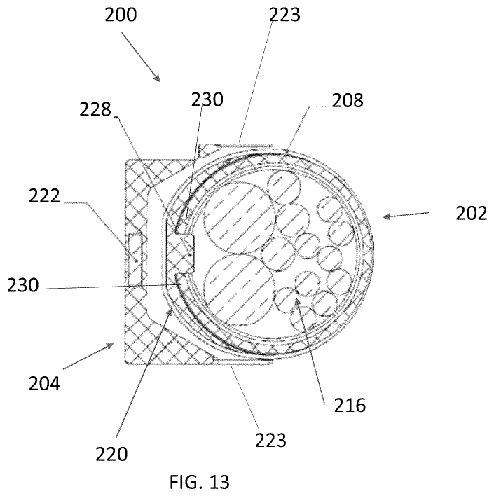

FIG. 13 illustrates a cross-sectional front view depicting the chain 200, in accordance with some embodiments of the present disclosure. As illustrated, the link 204 only partially encompasses or covers the tubing 202. This may cause the chain 200 to have a low-profile and allows greater flexibility for the chain 200 to turn within the gantry 56 (e.g., shown on FIG. 1). The tubing 202 may be secured within the recess 220 of the link 204. For example, the tubing 202 may be secured to the link 204 via a tightened wire tie 208 that compresses edges 230 of the tubing 202 against the tab 228. In some embodiments, the magnet 222 may be adjacent to the tab 228. Additionally, the cable bundle 216 may be disposed within the tubing 202. In some embodiments, the tubing 202 may be disposed between the extended portions 223 of the link 204.

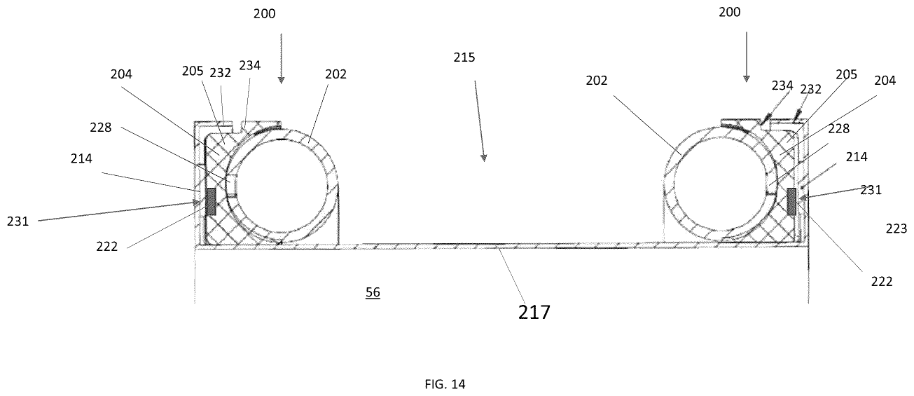

FIG. 14 illustrates a cross-sectional front view depicting a nested geometry of multiple chains 200 movably disposed within or against the gantry 56, in accordance with some embodiments of the present disclosure. A first chain 200 may be disposed at a first sidewall 214 of the gantry 56 and a second chain 200 may be disposed at a second sidewall 214 of the gantry 56. The chains 200 may be separated by a void 215 that may extend longitudinally along the gantry 56. In some embodiments, a surface 217 may extend between and adjacent to the sidewalls 214. The surface 217 may continuously extend along a length of the gantry 56 and in between the chains 200, as illustrated. The links 204 may be movably disposed against the sidewalls 214 such that the magnets 122 are adjacent to and/or in contact with ferrous material 231 disposed within the sidewalls 214. In some embodiments, the links 204 may be attached to the sidewalls 214 via the magnets 222 and ferrous material 231. Each of the sidewalls 214 may also include a flange 132 to mate with a step 234 of each housing 205 of the links 204 to assist in retaining the chains 200 against the sidewalls 214, as illustrated. The links 204 may move along the length of the sidewalls 214 and therefore along a length of the gantry 56. The tubing 202 may be attached to the links 204 via compression of the tabs 228 with the edges 230 of the tubing 202, as previously noted. Accordingly, each of the chains 200 is configured to move independently along the length of the gantry 56, in some examples.

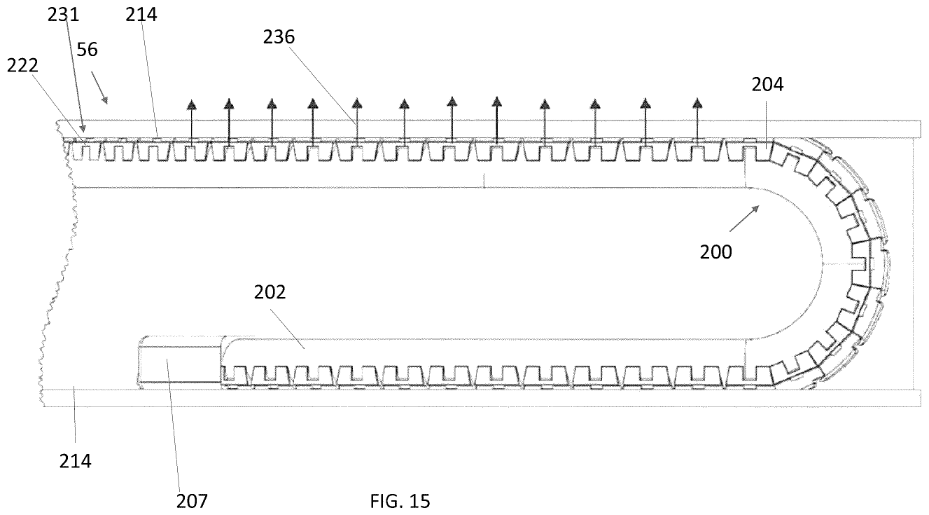

FIG. 15 illustrates the chain 200 movably disposed within the gantry 56, in accordance with embodiments of the present disclosure. As illustrated, the chain 200 may be in a horizontal position and the weight of the chain 200 is unsupported by the gantry 56, however, discrete magnetic forces indicated by directional arrows 236, support the weight of the chain 200 against the force of gravity. The directional arrows 236 indicate a direction that the chain 200 is pulled due to a magnetic attraction between the magnets 222 of the links 204 and the ferrous material 231 disposed within the sidewalls 214. For example, the tubing 202 is attached to the links 204 to form the chain 200 that is movably attached the sidewalls 214 of the gantry 56. The magnets 222 secure the chain 200 to the sidewalls 214 and in some embodiments, the chain 200 is pulled against the sidewalls 214 due to the magnetic forces. Additionally, the second end clamp 207 may be movably attached to the sidewalls 214 of the gantry 56, as previously noted.

As described herein, some benefits of the various embodiments include: (1) a chain with a low profile and improved flexibility; (2) a path of the chain follows a compound curve; (3) the chain functions independent of orientation due to a magnetic preload; and (4) the split tubing allows for installation around a cable bundle rather than threading the cable bundle through a completely enclosed tube, and also allows cable bundles to be pre-terminated with connectors rather than terminated after assembly.

What is claimed is:

1. A cable chain for use with a mechanical apparatus having an electrical component requiring electrical power, the cable chain comprising:
a flexible tubing;
a plurality of electrical cables disposed in the flexible tubing and coupled to the electrical component;
a plurality of links longitudinally spaced from each other along the tubing, each link including an inner surface forming a recess that receives the tubing, the each link being secured to the housing and having an outer surface in contact with a surface of the mechanical apparatus.

2. The cable chain of claim 1, wherein the flexible tubing includes a split tubing.

3. The cable chain of claim 2, wherein a split of the split tubing faces inner surfaces of the links.

4. The cable chain of claim 2, wherein:
each link includes a tab protruding from the inner surface of the link; and
the tab protrudes into a gap of a split portion of the split tubing.

5. The cable chain of claim 4, wherein the tubing is flexible.

6. The cable chain of claim 5, wherein the tubing is corrugated.

7. The cable chain of claim 6, further comprising a plurality of wire ties each disposed around a corresponding portion of the flexible tubing.

8. The cable chain of claim 1, further comprising a plurality of wire ties each disposed around a corresponding portion of the flexible tubing and securing the flexible tubing to a corresponding one of the links.

9. The cable chain of claim 8, wherein the flexible tubing includes a split tubing.

10. The cable chain of claim 9, wherein a split of the split tubing faces inner surfaces of the links.

11. The cable chain of claim 9, wherein:
each link includes a tab protruding from the inner surface of the link; and
the tab protrudes into a gap of a split portion of the split tubing.

12. The cable chain of claim 11, wherein the tubing is corrugated.

13. An imaging system comprising:
a c-arm having an x-ray transmitter;
a cable chain movably disposed in the c-arm and including:
a flexible tubing;
a plurality of electrical cables disposed in the flexible tubing and coupled to the x- ray transmitter;
a plurality of links longitudinally spaced from each other along the tubing, each link including an inner surface forming a recess that receives the tubing, the each link being secured to the housing and having an outer surface configured to move away from or be in contact with an outer surface of the c-arm as the flexible tubing moves relative to the c-arm.

14. The imaging system of claim 13, further comprising a plurality of wire ties each disposed around a corresponding portion of the flexible tubing and securing the flexible tubing to a corresponding one of the links.

15. The imaging system of claim 14, wherein the flexible tubing includes a split tubing.

16. The imaging system of claim 15, wherein a split of the split tubing faces inner surfaces of the links.

17. The imaging system of claim 15, wherein:
each link includes a tab protruding from the inner surface of the link; and
the tab protrudes into a gap of a split portion of the split tubing.

18. The imaging system of claim 17, wherein the tubing is corrugated.

19. The imaging system of claim 1, wherein the flexible tubing includes a split tubing and a split of the split tubing faces inner surfaces of the links.

20. The imaging system of claim 19, wherein:
each link includes a tab protruding from the inner surface of the link; and
the tab protrudes into a gap of a split portion of the split tubing.
